(19) United States
(12) Patent Application Publication
(10) Pub. No.: US 2021/0212722 A1
(43) Pub. Date: Jul. 15, 2021

(54) CHEST TUBE INSERTION DEVICE

(71) Applicant: AOK Innovations, LLC, Lexington, KY (US)

(72) Inventors: Jon Kiev, Lexington, KY (US); Justen England, Quincy, MA (US); Chris Harris, Cambridge, MA (US)

(21) Appl. No.: 17/144,939

(22) Filed: Jan. 8, 2021

Related U.S. Application Data
(60) Provisional application No. 62/959,691, filed on Jan. 10, 2020.

(51) Int. Cl. A61B 17/34; A61B 17/3209

(52) U.S. Cl. CPC A61B 17/3415; A61M 27/00; A61B 17/32093; A61B 17/3423

(57) ABSTRACT

A device to access interior body regions includes a grippable handle, a cannula having a proximal end and a distal end, a retractable obturator disposed within the cannula and extending prominently of the distal end of the cannula, the retractable obturator being spring-biased away from the proximal end of the cannula and having an axially-extending lateral slot that is open at a distal end of the obturator and an arrow-headed blade received within the slot of the obturator, the blade being exposed only when the obturator is urged toward the proximal end of the cannula, the blade having a tip with an edge-to-edge angle in a range of 30° to 50°. The cannula is selectively securable to a blade subassembly at a distal end of the grippable handle by a hub that can be decoupled from the device by pressing a cannula/hub release button of the blade subassembly.

### CROSS REFERENCE TO OTHER APPLICATIONS

This application claims benefit of the filing date of U.S. Provisional Patent Application No. 62/959,691, entitled, “Chest Tube Insertion Device,” filed on Jan. 10, 2020, the entire disclosure of which is hereby expressly incorporated by reference herein.

### FIELD OF THE DISCLOSURE

This disclosure relates generally to a device to facilitate insertion of a conduit into a bodily cavity and, more particularly, to a chest tube insertion aid with a protected, selectively-deployable blade and handle configuration to permit single-handed deployment.

### BACKGROUND

Chest tube insertion can be a labor-intensive exercise for the surgeon. Typically, the surgeon will make an incision into the skin and through the subcutaneous tissue, insert a Kelly clamp into the incision, dissect bluntly through the superficial fascia, intercostal muscle, and other subcutaneous tissue muscle, and into the intercostal space. With the Kelly clamp in a closed condition, the surgeon then punctures the pleura. The jaws of the Kelly clamp are then opened, stretching the tissue larger than a diameter of the chest tube to be inserted. Next, the surgeon uses an index finger to keep a track open to use the Kelly clamp for inserting the chest tube through the formed track and into the intrathoracic cavity. The Kelly clamp is then removed. This method of chest tube insertion is cumbersome and does not provide the surgeon with a free hand.

A chest tube insertion aid system and method are disclosed in U.S. Pat. Nos. 9,743,952 and 9,743,953, which are incorporated herein by reference in their entirety. Each system disclosed in these patents includes a safety needle assembly that is inserted through a handle hole at a proximal end of the system, and advanced into a blade assembly until the safety needle assembly is positioned at a distal end of the blade assembly. The combination of the safety needle assembly and blade assembly are then inserted through an obturator assembly, together forming a unit that is then connected to a dilator assembly. The safety needle has a cannula, a cannula tip at a distal end, a stylet, a stylet port, a stylet tip, and at a proximal end, a housing, a hub, and a connection means, such as a luer-type fitting. To confirm the safety needle assembly is inserted into the correct location within a patient, the connection means can be coupled to a fluid drainage device, such as a suction canister or syringe. While such a system is quite useful for certain situations, it is still somewhat cumbersome and, because of the length of the safety needle, can be a delicate instrument to maneuver, which can be incongruous with the force necessary to achieve percutaneous insertion.

### SUMMARY OF THE DISCLOSURE

There is a need for a handheld system that facilitates insertion of a chest tube into a patient that is sufficiently robust to withstand the rigors of forceful percutaneous insertion and that can be manipulated by a surgeon with one hand. By eliminating the safety needle assembly, the system of the present disclosure as a whole is significantly more robust. In one example, the system of the present disclosure includes a blade assembly having a preferably arrow-headed blade that is shielded by a slotted, generally conical distal end of an obturator. A hub at a proximal end of the obturator provides a point of securement to a handle. In one embodiment, the handle has a first, distal portion oriented co-axially with the obturator and blade assembly, and a second, proximal portion having an ergonomic shape that angles in a range of 5° to 30°, and preferably 15°, from the first, distal portion of the handle, facilitating a natural-feeling grip in the medical gloved hand of the surgeon, reminiscent of a pool cue. The ornamental appearance of the handle is not dictated solely by its function.

In an alternate embodiment, the handle has a first, distal portion oriented co-axially with the obturator and blade assembly, and a second, proximal portion that is angled approximately 90° from the first, distal portion of the handle, forming a pistol grip-like handle. The ornamental appearance of the handle of this alternate embodiment is also not dictated solely by its function.

The preferably arrow-headed blade of the blade assembly has a tip with an edge-to-edge angle in a range of approximately 30° to approximately 50°. The blade has a Rockwell hardness in a range of approximately 50 to approximately 55. The tip of the preferably arrow-headed blade of the blade assembly includes a 20° taper along the edges thereof.

### BRIEF DESCRIPTION OF THE SEVERAL VIEWS OF THE DRAWING

FIG. 1 is a front, right perspective view of a first exemplary chest tube insertion device assembled in accordance with the teachings of the present disclosure;

FIG. 2 is an exploded front, right perspective view of the chest tube insertion device of FIG. 1;

FIG. 3 is an exploded rear, right perspective view of the chest tube insertion device of FIG. 1;

FIG. 4 is a right elevation view of the chest tube insertion device of FIG. 1 with an obturator, hub, biasing spring, and cannula, illustrating the insertion device in an unshielded position;

FIG. 5 is a partial, enlarged right elevation view of a distal portion of the chest tube insertion device of FIG. 4;

FIG. 6 is a partial, enlarged right elevation view of the distal portion of the chest tube insertion device of FIG. 4, illustrating the obturator in an uncontracted, shielding condition in which an arrow-headed blade of the chest tube insertion device is not exposed;

FIG. 7 is a perspective view of a blade subassembly of the chest tube insertion device of FIG. 4;

FIG. 8 is an enlarged cross-sectional view of the chest tube insertion device of FIG. 1, taken along lines 8-8 of FIG. 1;

FIG. 9 is a front, right perspective view of a first exemplary blade assembly of the chest tube insertion device of FIG. 1 constructed in accordance with the teachings of the present disclosure;

FIG. 10 is a right elevation view of the blade assembly of FIG. 9;

FIG. 11 is a top view of the blade assembly of FIG. 9;

FIG. 12 is an enlarged view of a distal region of a second exemplary blade assembly of a chest tube insertion device constructed in accordance with the teachings of the present disclosure;

FIG. 13 is an enlarged view of a distal region of a third exemplary blade assembly of a chest tube insertion device constructed in accordance with the teachings of the present disclosure; and

FIG. 14 is a second exemplary chest tube insertion device with a second exemplary handle assembled in accordance with the teachings of the present disclosure.

### DETAILED DESCRIPTION OF THE PREFERRED EMBODIMENTS

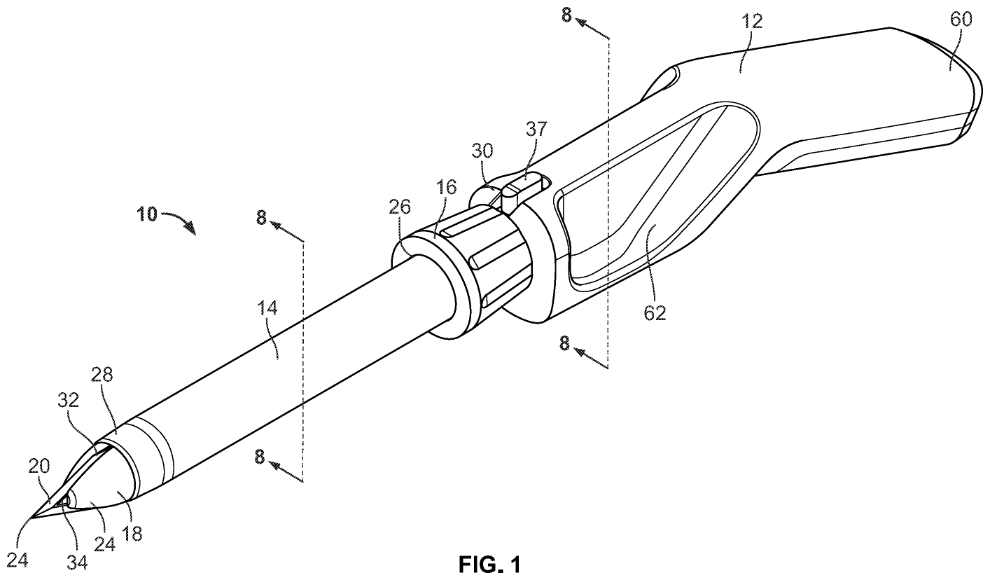

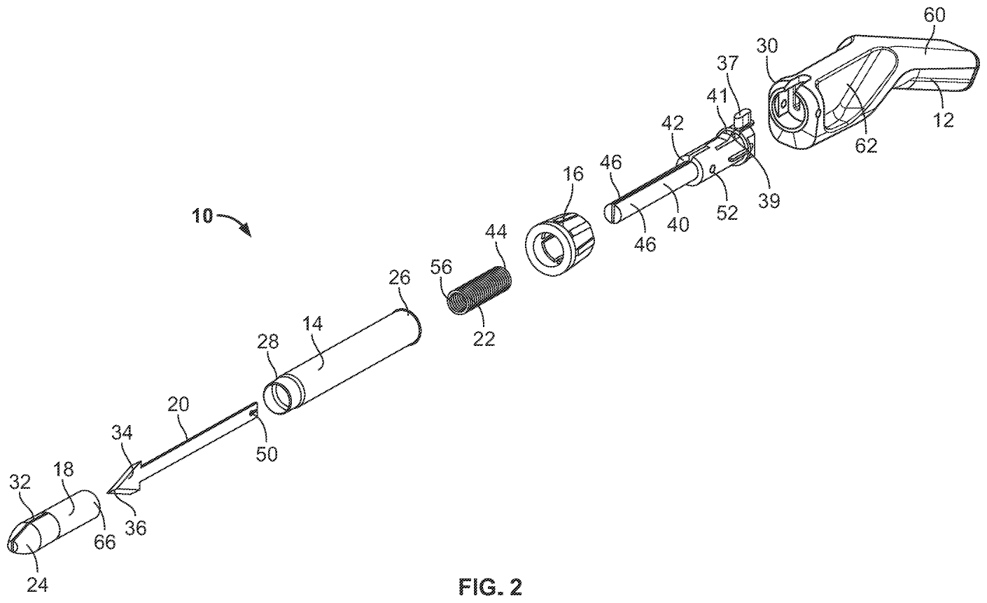

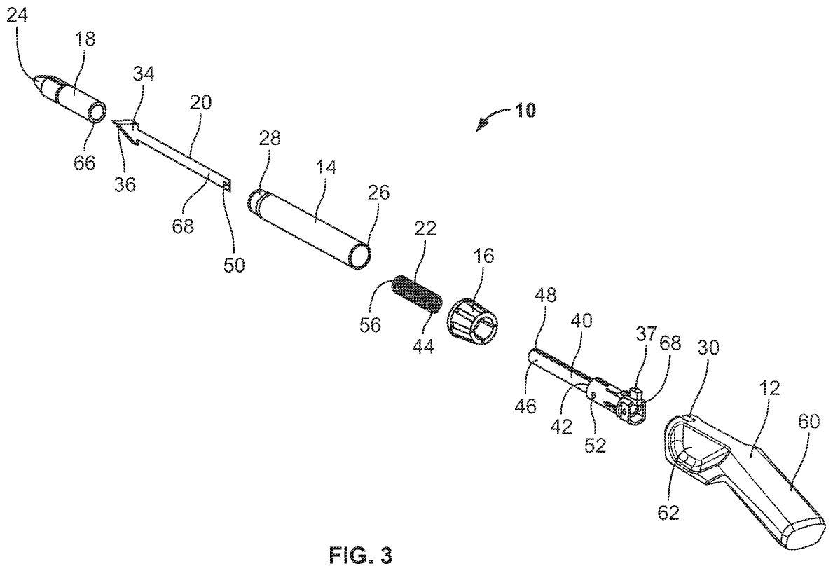

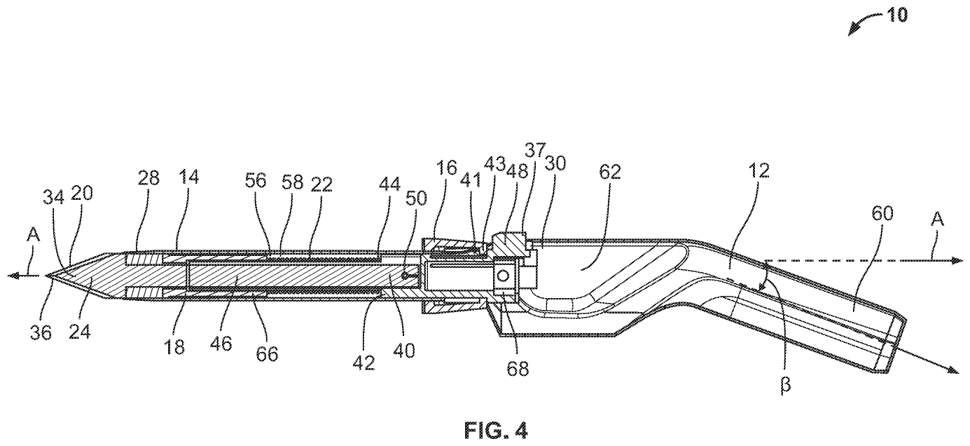

Referring generally to FIGS. 1-6, a chest tube insertion device 10 of the present disclosure includes a handle 12, a cannula 14, a hub 16, an obturator 18, an arrow-headed blade assembly 20, and a biasing spring 22. The protected, selectively-deployable blade and handle configuration of the device 10 permits single-handed deployment of a conduit into a bodily cavity. In particular, the device 10 facilitates insertion of a chest tube into a chest cavity of a patient. To deploy a chest tube, an operator first creates a pathway in the patient by pressing the device 10 onto an incision site of a patient. The obturator 18 protects the sharp edges of the blade assembly 20 when the device 10 is not in use. The obturator 18 of the insertion device 10 slides along an axis A of the device 10 relative to the blade assembly 20 and handle 12 to expose the blade assembly 20, as shown in FIGS. 1 and 4. The blade 20 cuts through skin and subcutaneous tissue of the patient as the device 10 is inserted. A tapered distal end 24 of the obturator 18 widens the incision made by the blade 20 as the device 10 is inserted further into the patient, to thereby accommodate the cannula 14 of the device 10, surrounding the obturator 18, within the expanded incision in the patient and toward the deployment site. When ready, the operator may actuate a tab or cannula/hub release button 37 of a blade subassembly 40 adjacent the handle 12, which de-couples the cannula 14 and the hub 16 from the remainder of the device 10, thereby permitting the user to withdraw (by pulling the handle 12 along the axis A in a direction away from the patient) the obturator 18 and blade assembly 20 from the patient and insert a chest tube (not shown) through the hub 16 and cannula 14, which remains in the patient as a conduit.

As shown in FIGS. 1-4, the cannula 14 includes a proximal end 26 and a distal end 28 and extends from a distal end 30 of the handle 12. The retractable obturator 18 is disposed within the cannula 14 and extends prominently from the distal end 28 of the cannula 14. The retractable obturator 18 is spring-biased away from the proximal end 26 of the cannula 14 and has an axially-extending lateral slot 32 that is open at the distal end 24 of the obturator 18. An arrow-headed blade 34 of the blade assembly 20 is received within the slot 32 of the obturator 18 and is exposed only when the obturator 18 is urged toward the proximal end 26 of the cannula 14. The blade 34 has a tip 36 with an edge-to-edge angle in a range of approximately 30° to approximately 50° with a Rockwell hardness in a range of approximately 50 to approximately 55. The hub 16 receives a portion of the cannula 14 and releasably secures the cannula 14 to the handle 12.

The blade subassembly 40 will now be described in further detail, with reference to FIGS. 1-6. The blade subassembly 40 includes an annular shoulder 42, against which rests a proximal end 44 of the biasing spring 22, and an elongate cylindrical region 46. The blade subassembly 40 is removably coupled to the handle 12 at the distal end 30 of the handle 12 and is fixedly attached to the blade assembly 20. The elongate cylindrical region 46 includes an axial slot 48 that is sized to receive the blade assembly 20. The blade assembly 20 may be removably coupled to the blade subassembly 40 via a pin disposed through both an aperture 50 formed in the blade assembly 20 and an aperture 52 formed in the blade subassembly 40.

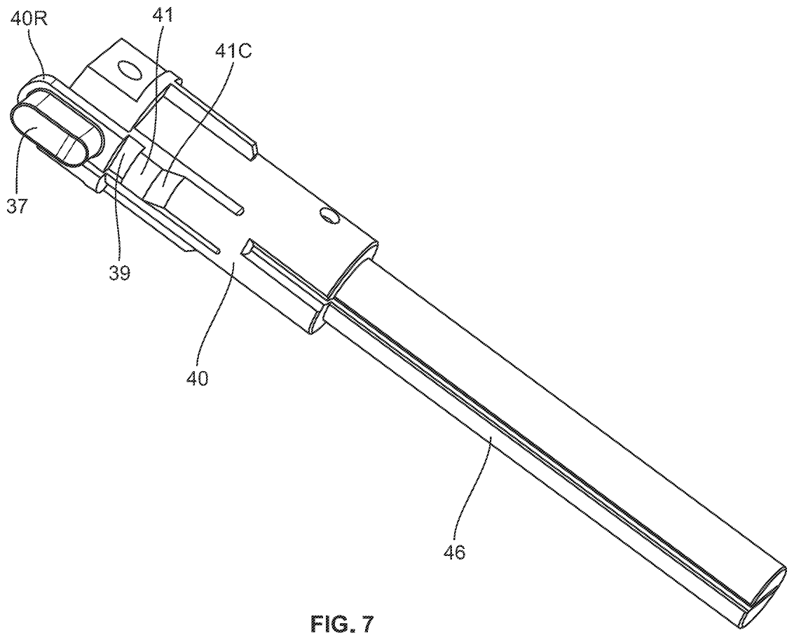

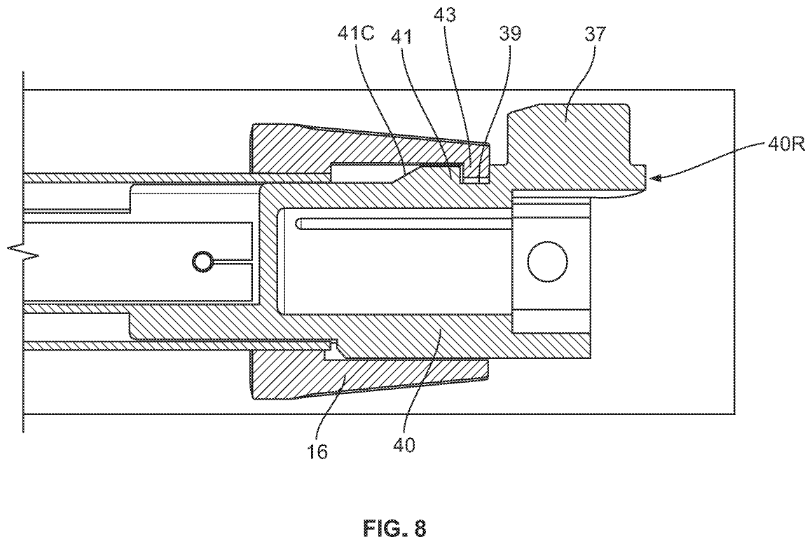

The blade subassembly 40 further includes a cantilevered release member 40R at a proximal end thereof, illustrated in FIGS. 7 and 8. The cantilevered release member 40R includes the cannula/hub release button 37, a raised ridge 41 spaced distally from cannula/hub release button 37, and an arcuate slot 39. The arcuate slot is disposed between the raised ridge 41 (which is immediately distally of the arcuate slot 39) and the cannula/hub release button 37, and is sized to receive a complementary locking lip 43 extending from an interior of the hub 16, as shown in FIG. 8. A chamfered surface 41C is provided along a leading (distal) end of the raised ridge 41 that facilitates insertion of the hub 16 and cannula 14, by serving as a ramp that the locking lip 43 rides along so it can pass over the raised ridge 41 and into the arcuate slot 39. An audible and/or tactile click may be experienced to confirm that the locking lip 43 of the hub 16 is securely received within the arcuate slot 39 of the blade subassembly 40. The cannula/hub release button 37 advantageously provides the surgeon with a reliable, one-handed trigger mechanism to initiate decoupling of the cannula 14 and hub 16 from the rest of the device 10, as compared to earlier chest tube insertion aid systems that required rotation of the handle relative to the cannula, or required sliding against a friction fit between the cannula and obturator assembly.

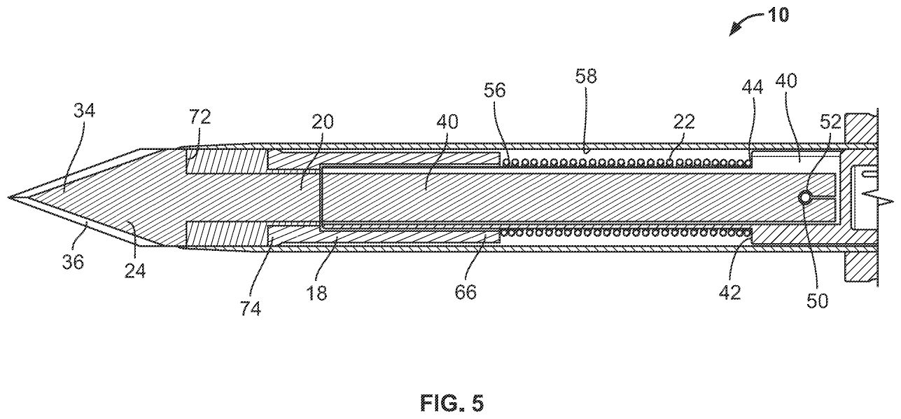

The distal end 24 of the obturator 18 is slotted to receive the arrow-headed portion 34 of the blade assembly 20. When the device 10 is in an unshielded position as depicted in FIGS. 4 and 5, the distal tip 36 of the blade assembly 20 extends through the slot 32 formed in the distal end 24 of the obturator 18. The distal end 24 of the obturator 18 is generally conical or tapered to facilitate passing the device 10 through an incision site, widening the cuts in skin and subcutaneous tissue made by the blade assembly 20 to form a pathway for the conduit (e.g., chest tube). The obturator 18 is retained by the blade assembly 20 so that when not subject to a resistive force from surrounding tissue, the obturator 18 slides away from the handle 14 to sufficiently shield the blade 20.

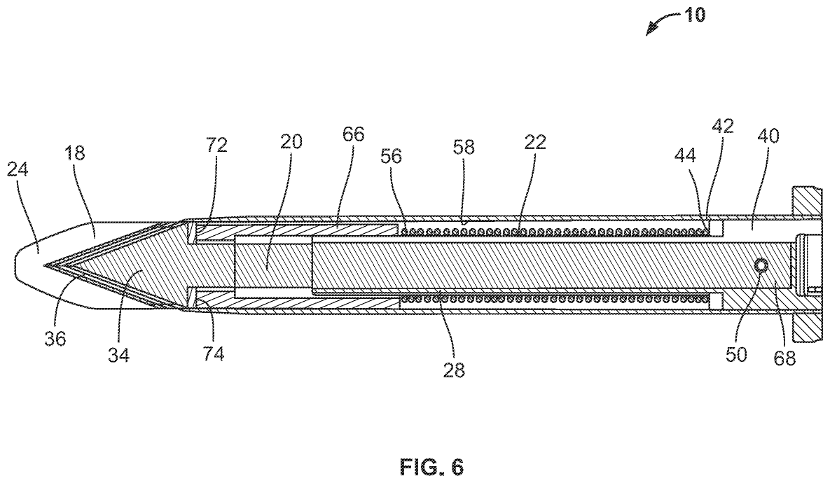

Turning to FIGS. 5 and 6, the spring 22 is a coil spring having a stiffness or spring constant selected to resist contraction, and thereby prevent retraction of the distal shielding region 24 of the obturator 18 proximally into the cannula 14, upon incidental contact with the distal shielding region 24 of the obturator 18. The spring 22 permits spring contraction, thereby allowing proximal retraction of the distal shielding region 24 of the obturator 18, and exposure of the underlying tip portion 36 of the arrow-headed blade 34. At least about eight pounds of force applied to the distal shielding region 24 of the obturator 18 exposes the underlying tip portion 36 of the blade assembly 20. The obturator 18 rests against a distal end 56 of the biasing spring 22. An inner wall of the cannula 14, the elongate cylindrical region 28 of the blade subassembly 40, and the annular shoulder 42 define an annular channel 58 for the biasing spring 22.

As illustrated in FIG. 4, the first exemplary device 10 includes a first exemplary handle 12. The handle 12 has a first, distal portion 30 oriented co-axially with the obturator 18 and blade assembly 20 along the A axis, and a second, proximal portion 60 that is oriented at an angle β relative to the A axis. The proximal portion 60 has an ergonomic shape in which the angle β is in a range of approximately 5° to approximately 30°, and preferably approximately 15°, from the first, distal portion 30 of the handle 12. The angled configuration of the handle 12 facilitates a natural-feeling grip in the medical gloved hand of the surgeon, reminiscent of a pool cue. In the distal portion 30 of the handle 12, first and second parallel grooves 62 are formed to receive a thumb and index finger, and the proximal portion 60 of the handle 12 is configured and shaped to rest comfortably in a palm of the hand of the surgeon. The grooves 62 and proximal portion 60 of the handle 12 may include dimples, grooves, or other textures on the handle's surface to aid in gripping the handle 12. The ornamental appearance of the handle 12 is not dictated solely by its function.

In FIGS. 4 and 5, the arrow-headed blade 34 of the insertion device 10 is illustrated in an unshielded configuration. In the unshielded configuration, the spring 22 is in a compressed position between a proximal end 66 of the obturator 18 and the shoulder 42 of the blade subassembly 40. The arrow-head blade 34 of the blade assembly 20 extends beyond the distal region 24 of the obturator 18, exposing the sharp edges 36 of the blade 20. When the insertion device 10 moves from the shielded to the unshielded position, the edges of the blade assembly 20 extend through the slot 32 formed in the distal region 24 of the obturator 18 as the obturator 18 moves in a proximal direction toward the handle 12 along the axis A. Also shown in FIG. 4, the aperture 50 in a proximal end 68 of the blade assembly 20 aligns with the aperture 52 of the blade subassembly 40 so that a pin or other fastening device may secure the blade assembly 20 to the blade subassembly 40.

In FIG. 6, the arrow-headed blade 34 of the insertion device 10 is shown in a shielded configuration. In the shielded configuration, the spring 22 is expanded to bias the obturator 18 away from the handle 12 to cover the edges 36 of the blade assembly 20. The obturator 18 is in an uncontracted, shielding position in which the arrow-headed blade 34 of the chest tube insertion device 10 is not exposed. As previously described, the obturator 18 remains attached to the device 10 when the arrow-headed blade 34 of the device 10 is in the shielded configuration. Although the spring 22 biases the obturator 18 away from proximal end 68 of the blade assembly 20, a back edge 72 of the blade 34 keeps the obturator 18 attached to the device 10. In particular, the obturator 18 includes a shoulder 74 formed in an interior surface of the obturator 18 that protrudes inwardly to catch against the back edge 72 of the blade 34. As such, when the device is in the shielded position, the expanded coil 22 biases the shoulder 74 of the obturator 18 against the back edge 72 of the blade 34, retaining the obturator to the device 10.

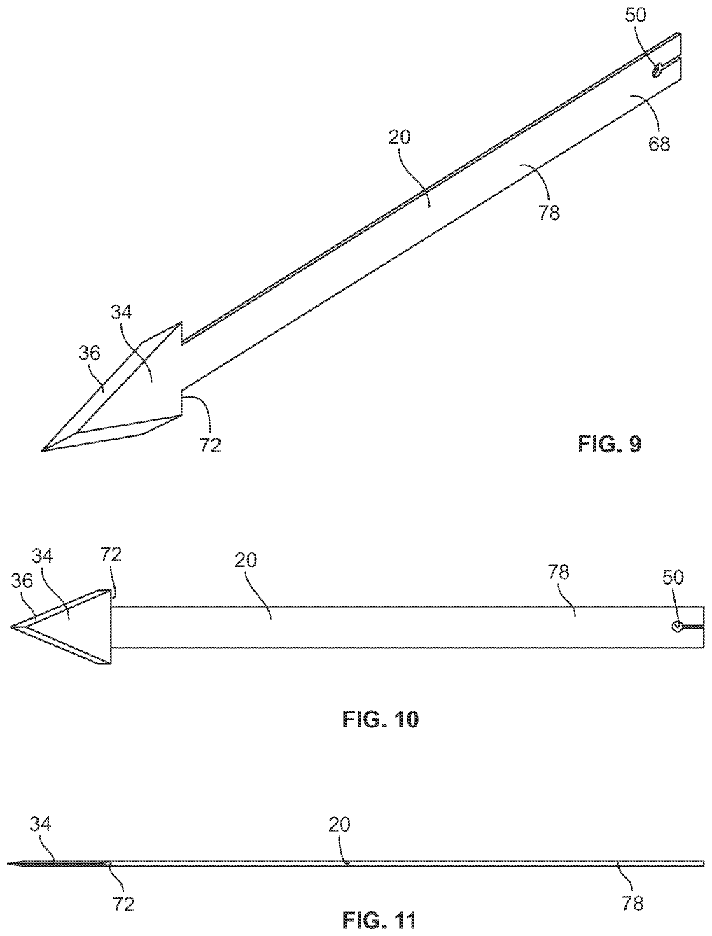

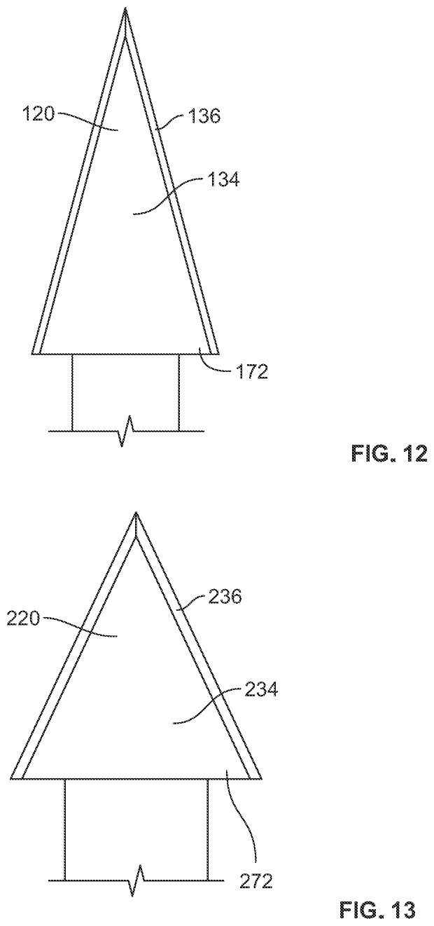

In FIGS. 9-11, the first exemplary blade assembly 20 is shown in more detail. The arrow-headed blade 34 of the blade assembly 20 includes a distal point and the back edge 72 adjacent to a flat body 78. The flat body 78 of the blade assembly 20 is sized to slide through the cylindrical body 46 of the blade subassembly 40. The blade 34 has a Rockwell hardness in a range of approximately 50 to approximately 55. The tip 36 of the preferably arrow-headed blade 34 of the blade assembly 24 includes an approximately 20° taper along the edges thereof. The blade 34 of the blade assembly 20 has a preferably arrow-headed edge 36 with an edge-to-edge angle in a range of approximately 30° to approximately 50°. In particular, an exemplary blade assembly 120 of FIG. 12 has an edge-to-edge angle of approximately 30°, and an exemplary blade assembly 220 of FIG. 13 has an edge-to-edge angle of approximately 50°. Any of the exemplary blade assemblies 20, 120, 220 may be use with the insertion device 10 described herein and the second and third exemplary blade assemblies 120, 220 are configured to couple to the remainder of the device 10 as previously described with respect to the first exemplary blade assembly 20. The blade assembly 20, 120, 220 may be permanently fixed or removably attached to the blade sub assembly 24 so that the blade assembly 20, 120, 220 remains stationary relative to the handle 12, cannula 14, and hub 16, of the device 10.

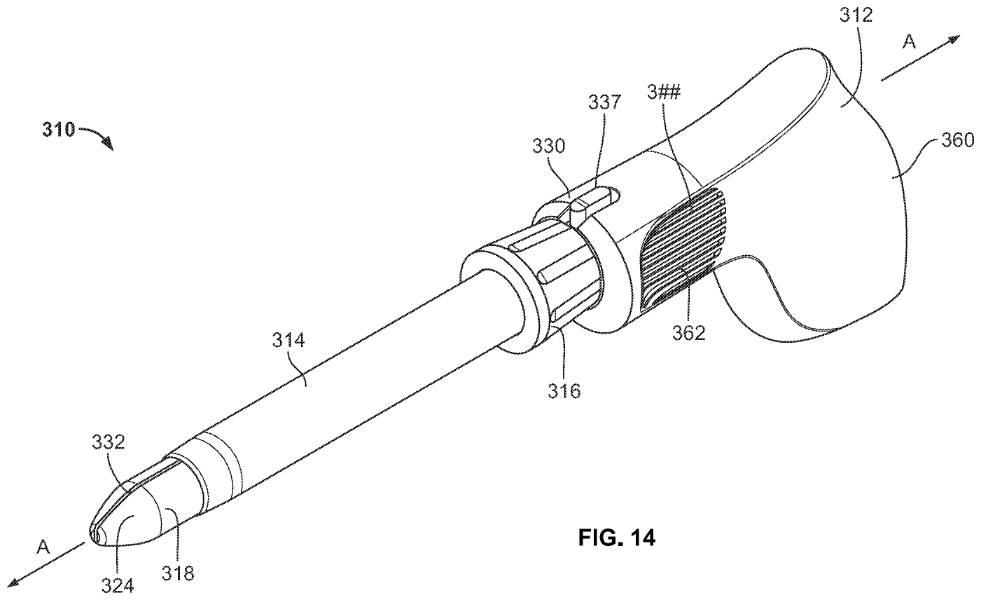

FIG. 14 illustrates a second exemplary insertion device 310 assembled in accordance with the teachings of the present disclosure. The insertion device 310 of FIG. 14 is similar to the insertion device 10 described above in FIGS. 1-8, except that the second exemplary insertion device 310 is equipped with a different handle 312. Thus for ease of reference, and to the extent possible, the same or similar components of the second exemplary insertion device 310 will retain the same reference numbers as outlined above although the reference numbers will be increased by 300.

The second exemplary handle 312 of FIG. 14 forms a pistol grip-like handle. The handle 312 has a first, distal portion 330 oriented co-axially with an obturator 318 and a blade assembly (hidden from view). The handle 312 has a second, proximal portion 360 that is angled approximately 90° from the first, distal portion 330 of the handle 312. The proximal portion 360 is rounded to comfortably rest in a palm of a surgeon's hand and first and second parallel grips (e.g., contours, indentations, or grooves) 362 are oriented so that a surgeon's thumb and index finger naturally rest on either side of the distal portion 330 of the handle 312. Each one of the grips 362 includes a textured surface in the form of a plurality of parallel grooves. However, in other examples, the textured surface may include dimples, non-parallel grooves, or other textures on the handle's surface to aid in gripping the handle 312. The ornamental appearance of the handle 312 of this alternate embodiment is also not dictated solely by its function.

The disclosed exemplary arrangements of the chest tube insertion device 10, 310 provide robust construction and simplified design. By comparison to other known instruments, the present arrangements are simplified in design and do not include delicate needles and injection devices. When using the disclosed device 10, 310 with a patient, a surgeon can make an incision and widen a pathway for a chest tube (or other conduit) without fear of breaking or damaging any delicate components of the insertion device in the patient. Additionally, the device may be used with a wide range of patients in terms of age, body size, and weight because the device 10, 310 adjusts to the force applied by the surgeon for entry into a patient's body cavity. For example, the blade assembly 20, 120, 220, 320 will be exposed only to the extent necessary for make an incision.

Additionally, the simplified design of the exemplary arrangements of the insertion device 10, 310 facilitates insertion of a conduit, such as a chest tube, within a bodily cavity. In the illustrated arrangements, a surgeon may make an incision and pathway for a chest tube without needing a Kelly clamp or using their index finger or other instrument to keep a pathway open in the patient. In this way, a surgeon has a free hand to hold a chest tube and insert the chest tube into the pathway of the patient without requiring the help of an assistant. The ergonomic exemplary handles 12, 312 described and illustrated herein permit the surgeon to comfortably grip the device and apply sufficient force to unshield the blade assembly 20, 120, 220, 320 and form a pathway in the patient. Once the device 10, 310 is fully inserted, the surgeon can depress the cannula/hub release button 37, 337 to decouple the cannula 14, 314 and hub 16, 316 from the rest of the respective device 10, 310. The surgeon then pulls the handle 60, 360 in a direction away from the patient along the axis A, removing the entirety of the device 10, 310 with the exception of the cannula 14, 314 and hub 16, 316, which remain in place as a conduit into the patient's chest cavity through which a chest tube may then readily be inserted.

The figures and description provided herein depict and describe preferred embodiments of a chest tube insertion device for purposes of illustration only. One skilled in the art will readily recognize from the foregoing discussion that alternative embodiments of the components illustrated herein may be employed without departing from the principles described herein. Thus, upon reading this disclosure, those of skill in the art will appreciate still additional alternative structural and functional designs for the chest tube insertion device. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the methods and components disclosed herein without departing from the spirit and scope defined in the appended claims.

What is claimed is:

1. A device to access interior body regions comprising:
a grippable handle;
a cannula extending from a distal end of the grippable handle, the cannula having a proximal end and a distal end;
a retractable obturator disposed within the cannula and extending prominently of the distal end of the cannula, the obturator being spring-biased away from the proximal end of the cannula and having an axially-extending lateral slot that is open at a distal end of the obturator; and
an arrow-headed blade received within the slot of the obturator, the blade being exposed when the obturator is urged toward the proximal end of the cannula, the blade having a tip with an edge-to-edge angle in a range of approximately 30° to approximately 50° and the blade having a Rockwell hardness in a range of approximately 50 to approximately 55.

2. The device of claim 1, wherein the tip of the arrow-headed blade includes an approximately 20° taper along an edge of the tip.

3. The device of claim 1, wherein the grippable handle is positioned such that a palm rest of the grippable handle is oriented parallel to the blade.

4. The device of claim 1, wherein the obturator is spring-biased by a coil spring having a stiffness sufficient to resist movement of the obturator.

5. The device of claim 1, further comprising a blade subassembly disposed at the distal end of the grippable handle, the blade subassembly including a shoulder against which the obturator is spring biased and a hub secured to the cannula and selectively securable to the blade subassembly.

6. The device of claim 5, wherein the blade subassembly includes a cantilevered release member at a proximal end thereof, including a cannula/hub release button, a raised ridge spaced distally from cannula/hub release button, and a slot that is sized to receive a complementary locking lip on an interior of the hub, the arcuate slot disposed between the raised ridge and the cannula/hub release button.

7. The device of claim 6, further including a chamfered surface along a distal end of the raised ridge.

8. A method of inserting a tube into a bodily orifice, comprising:
urging, against skin to be penetrated, a distal end of a device including grippable handle; a cannula extending from a distal end of the grippable handle, the cannula having a proximal end and a distal end; a retractable obturator disposed within the cannula and extending prominently of the distal end of the cannula, the obturator being spring-biased away from the proximal end of the cannula and having an axially-extending lateral slot that is open at a distal end of the obturator; and an arrow-headed blade received within the slot of the obturator, the blade being exposed when the obturator is urged toward the proximal end of the cannula, the blade having a tip with an edge-to-edge angle in a range of approximately 30° to approximately 50° and the blade having a Rockwell hardness in a range of approximately 50 to approximately 55, using a force that exceeds a restoring force of the spring-biased obturator until the obturator retracts sufficiently to expose the blade, thereby forming an incision through the skin;
continuing to urge the device though the skin and subcutaneous tissue, thereby dissecting the tissue, until a location where a tube is to be inserted is reached;
de-coupling the cannula from the handle;
pulling the grippable handle in a direction away from the cannula and withdrawing the device, leaving the cannula in the formed opening through the skin and subcutaneous tissue; and
inserting a tube through the cannula.

9. The method of claim 8, wherein decoupling the cannula from the handle includes by depressing a cannula/hub release button of a blade subassembly selectively secured to the cannula by a hub at the distal end of the grippable handle, the cannula/hub release button disposed on a cantilevered release member of the blade subassembly, the hub including a locking lip on an interior thereof that is selectively received in a complementary slot of the cantilevered release member distally of the cannula/hub release button and proximally of a raised ridge, the locking lip of the hub clearing the raised ridge upon depression of the cannula/hub release button.

10. A device to access interior body regions comprising:
a grippable handle;
a cannula extending from a distal end of the grippable handle, the cannula having a proximal end and a distal end;
a retractable obturator disposed within the cannula and extending prominently of the distal end of the cannula, the obturator being spring-biased away from the proximal end of the cannula and having an axially-extending lateral slot that is open at a distal end of the obturator; and
a solid blade received within the slot of the obturator, the blade being exposed when the obturator is urged toward the proximal end of the cannula, the blade having a tip with an edge-to-edge angle in a range of approximately 30° to approximately 50°.

11. The device of claim 10, wherein the blade has a Rockwell hardness in a range of approximately 50 to approximately 55.
